(19) United States
(12) Patent Application Publication
(10) Pub. No.: US 2022/0047168 A1
(43) Pub. Date: Feb. 17, 2022

(54) DEVICE FOR OPTOACOUSTIC IMAGING AND CORRESPONDING CONTROL METHOD

(71) Applicant: iThera Medical GMBH, Munich (DE)

(72) Inventors: Stefan Morscher, Schnaitsee (DE); Josef Konradl, Odelzhausen (DE)

(21) Appl. No.: 17/276,784

(22) PCT Filed: Oct. 24, 2019
(86) PCT No.: PCT/EP2019/079026

§ 371 Date: Mar. 16, 2021

(30) Foreign Application Priority Data
Oct. 29, 2018 (EP) 18203095.7

(51) Int. Cl. A61B 5/00

(52) U.S. Cl. CPC A61B 5/0095

(57) ABSTRACT

The invention relates to a device for optoacoustic imaging of an object and a method for controlling such a device. An irradiation unit is configured to emit electromagnetic radiation and to irradiate an object with the electromagnetic radiation, and at least one reference element is arranged such that a part of the electromagnetic radiation emitted by the irradiation unit impinges on the reference element. The reference element is configured to emit first acoustic waves in response to the impinging electromagnetic radiation. Further, a detection unit is configured to detect first acoustic waves emitted by the reference element and second acoustic waves emitted by the object in response to irradiating the object with the electromagnetic radiation. The detection unit is further configured to generate an according first detection signal and second detection signal, respectively. A processing unit is configured to correct, in particular to normalize, the second detection signal using the first detection signal to obtain a corrected second detection signal, and to generate image information regarding the object based on the corrected, in particular normalized, second detection signal.

The present invention relates to a device for optoacoustic imaging of an object and a method for controlling such a device.

Optoacoustic signal generation is based on the optoacoustic effect, according to which ultrasonic waves are generated due to absorption of electromagnetic radiation by an object, e.g. a biological tissue, and a subsequent thermoelastic expansion of the object. Excitation radiation, for example non-ionizing laser light or radiofrequency radiation, can be pulsed radiation or continuous radiation with varying amplitude or frequency. This provides optical contrast at resolutions not limited by diffraction in the optical regime, giving rise to substantially improved resolution at depth. Because the absorption of electromagnetic radiation in tissue is usually a function of physiological properties, for example hemoglobin concentration or oxygen saturation, the optoacoustic effect is predestinated for utilization in medical imaging.

It is an object of the invention to improve optoacoustic imaging of an object, in particular to provide particularly reliable image information regarding the object, e.g. devoid of artifacts depending on imaging conditions and/or imaging system performance variations.

This objective is achieved by the device for optoacoustic imaging of object and the method for controlling such a device according to the independent claims.

The device according to an aspect of the invention comprises an irradiation unit configured to emit electromagnetic radiation and to irradiate an object with the electromagnetic radiation, and at least one reference element arranged such that a part of the electromagnetic radiation emitted by the irradiation unit impinges on the reference element. The reference element is configured to emit first acoustic waves in response to the impinging electromagnetic radiation. Further, a detection unit is configured to detect first acoustic waves emitted by the reference element and second acoustic waves emitted by the object in response to irradiating the object with the electromagnetic radiation. The detection unit is further configured to generate an according first detection signal and second detection signal, respectively. A processing unit is configured to correct, in particular to normalize, the second detection signal using the first detection signal to obtain a corrected second detection signal, and to generate image information regarding the object based on the corrected, in particular normalized, second detection signal.

A method according to another aspect of the invention comprises the steps of: controlling an irradiation unit to emit electromagnetic radiation and to irradiate the object with the electromagnetic radiation; controlling a detection unit to detect first acoustic waves emitted by a reference element arranged such that a part of the electromagnetic radiation emitted by the irradiation unit impinges on the reference element, the first acoustic waves being emitted by the reference element in response to the impinging electromagnetic radiation, and to generate an according first detection signal; controlling the detection unit to detect second acoustic waves emitted by the object in response to irradiating the object with the electromagnetic radiation and to generate an according second detection signal; and controlling a processing unit to correct, in particular normalize, the second detection signal using the first detection signal to obtain a corrected second detection signal, and to generate image information regarding the object based on the corrected, in particular normalized, second detection signal.

A preferred aspect of the invention is based on the approach of calibrating a device for optoacoustic imaging of an object, in particular a detection unit thereof, by providing at least one reference element which is irradiated with electromagnetic radiation generated and emitted by an irradiation unit. By using a first detection signal which is generated by the detection unit and corresponds to first acoustic waves generated in the at least one reference element in response to impinging electromagnetic radiation as a reference signal, a processing unit corrects, in particular normalizes, a second detection signal which is generated by the detection unit and corresponds to second acoustic waves generated in the object in response to impinging electromagnetic radiation. The corrected second detection signal is then used to generate image information regarding the object, e.g. in form of a calibrated optoacoustic absorption map.

Preferably, the first detection signal characterizes at least a part of a propagation path between the detection unit and the object, in particular a surface of the object, along which the first acoustic waves, and preferably also the second acoustic waves, propagate. In other words, the first detection signal comprises pathway information relating to, preferably characteristic, properties of the device, in particular its components, e.g. the irradiation unit, the detection unit and/or a coupling medium used to acoustically couple the detection unit to the object, and/or the object. For example, the first detection signal contains information on fluctuations of the intensity of the emitted electromagnetic radiation, changes in device materials and/or the object through e.g. temperature fluctuations, humidity, aging and/or the like. The first detection signal may for example contain information relating to the sound velocity of acoustic waves in the device and/or information relating to the attenuation of acoustic waves and/or the emitted electromagnetic radiation in the device. Accordingly, the first detection signal may be used to calibrate the device in-situ, in particular its components. By this means, artifacts in quantities derived from the second detection signal may be removed or at least mitigated such that the reliability and/or quality of the obtained image information is increased considerably.

The at least one reference element is preferably configured, in particular arranged, to emit the first acoustic waves towards the detection unit. By matching the size of the at least one reference element to a frequency range of the detection unit, e.g. an ultrasound transducer or an array thereof, the amplitude of the generated optoacoustic signal may be increased, allowing a particularly high signal to noise ratio (SNR). In principle, the at least one reference element can be made of any material absorbing the electromagnetic radiation, e.g. colored glass, polystyrene or polyethylene, which provide high durability, availability and control over the shape of the reference element. Especially in cases where there is no coupling medium except ultrasound gel, or the coupling material is liquid (e.g. water, D₂O) these materials can be of advantage, since they can be more precisely arranged and/or suffer less e.g. of diffusion of a coloring agent into the coupling medium which may lead to reduced light transmission towards the object. The sound propagation properties inside the reference element is of secondary interest, since any disadvantage in this aspect leading to reduced signal amplitude or reliability can preferably be counter-weighted by increased absorptivity.

Optoacoustic emission can be advantageously maximized by using a material exhibiting high absorption, e.g. in a range between 0.01 cm⁻¹ and 5 cm⁻¹. Preferably, the material used is chosen depending on the light flux at its location relative to the detection unit to enable a good detection SNR on the one hand while not saturating the acoustic detector and its acquisition electronics on the other hand. Light scattering property of the at least one reference element in a range between 5 and 20 cm⁻¹ is beneficial to enable more homogenous light absorption resulting in evenly distributed transient heating and thus acoustic wave generation that is in accordance with the signal expected by an absorber of the size. Without scattering the object would behave like it was only a shell, so that the signal would feature two sharp peaks (the edges) rather than a continuous, lower frequency signal that is desired by the “size matching to frequency”.

A variety of different shapes and sizes for the at least one reference element are possible, e.g. spherically or semispherically shaped reference elements, wherein the size of the reference element determines the frequency of the emitted signal. Due to its nature resulting from a transient heating process arising from light absorption, the shape of the acoustic wave follows the surface of the emitting structure. As a result, a spherical object results in a spherical wave, while planar surfaces will emit largely planar waves following the direction of the planar surface. Preferably, this effect is utilized to perform wave shaping in a manner to maximize the optoacoustic signal of the at least one reference element propagating on a primary, in particular direct, propagation path to the detection unit, while minimizing the optoacoustic signal potentially propagating along further propagations paths, in particular into any other direction that would incur a delayed time of arrival at the detection unit and cause a detrimental influence on the resulting image, because they might overlap with optoacoustic signals emitted from the tissue of interest.

In summary, the invention provides improved optoacoustic imaging of an object, in particular a particularly reliable correction of second detection signals corresponding to acoustic waves generated in the imaged object.

Preferably, the at least one reference element has a fixed position and/or orientation with respect to the detection unit and/or the irradiation unit and/or the object. Alternatively or additionally, the at least one reference element has a fixed position between the detection unit and/or the irradiation unit, on the one hand, and the object to be imaged, on the other hand.

Preferably, the at least one reference element is arranged in a, preferably fixed, position, such that a first part of the electromagnetic radiation emitted by the irradiation unit impinges on the reference element, while a second part of the electromagnetic radiation emitted by the irradiation unit, preferably simultaneously or substantially simultaneously, impinges on the imaged object.

Preferably, the device comprises, e.g. in the region of a distal end, a preferably handheld probe, which is designed for being brought into contact with the imaged object, for example human tissue, in order to obtain optoacoustic images from a region of interest in the object. The at least one reference element is preferably integrated in or an integral component of the probe.

By means of at least one of the aforementioned preferred embodiments, both first acoustic waves emitted by the at least one reference element and second acoustic waves emitted by the imaged object are generated, preferably simultaneously or substantially simultaneously, during optoacoustic imaging of the object.

As a result, first acoustic waves and according first detection signals can be detected and obtained, respectively, and used to correct the second detection signals during optoacoustic imaging of the object.

Advantageously, correcting the second detection signals obtained from the second acoustic waves emitted by the imaged object is not limited to a separate calibration process prior to or after the optoacoustic imaging process or during an interruption of the optoacoustic imaging process. Rather, it is possible to generate and detect first acoustic waves emitted by the reference element(s) whenever it is desired or necessary during the imaging process without interrupting same. For example, it is possible to obtain first detection signals in response to each pulse of electromagnetic radiation emitted by the irradiation unit and to use the obtained first detection signals for correcting respective second detection signals obtained for each of the pulses.

Further, as the device or probe comprises the reference element(s), additional or separate items, e.g. a separate calibration element or the like to be placed in front of the device or probe during a separate calibration process, is dispensable.

In a preferred embodiment, the device comprises a memory unit configured to store the first detection signals and/or the obtained image information. This is particularly useful if the device is used in or as a part of a hybrid system comprising a conventional ultrasound device, because the information stored in the memory unit can then be utilized for correction of relating ultrasound signals.

Alternatively or additionally, the memory unit may comprise additional calibration information which may be considered by the processing unit when the second detection signal is obtained. For example, the additional calibration information comprises one or more weighting factors applicable to the first detection signal and/or one or more calculation rules for calculating the corrected second signal based on the first and second detection signal. Alternatively or additionally, the additional calibration information comprises temporal information of acoustic waves emitted towards the object by the detection unit in a pulse/echo-mode for ultrasound imaging of the device. For instance, using a (known) distance of the detection unit to the at least one reference element it is possible to determine acoustic properties, in particular the speed of sound in the coupling medium, and use this to correct the second detection signals and/or detect the necessity of maintenance of the device. By this means, a particularly comprehensive in-situ calibration of the device can be achieved.

In another preferred embodiment, the at least one reference element is arranged such that a first time of flight, which is required by the first acoustic waves emitted by the reference element to impinge on the detection unit, is different to a second time of flight, which is required by the second acoustic waves emitted by the object to impinge on the detection unit. Preferably, the first time of flight corresponds to the time needed by the first acoustic waves to propagate along the primary propagation path from the reference element to the detection unit and/or the second time of flight corresponds to the time needed by the second acoustic waves to propagate along a secondary propagation path from the object to the detection unit.

By this arrangement, superposition of the first detection signal containing information on the propagation path, in particular the primary propagation path, onto the second detection signal containing information relating to the subject is reliably avoided. In particular, by discerning the first and the second time of flight, the first detection signal and the second detection signal can be easily and reliably distinguished from each other. This allows for a particularly reliable correction of the second detection signals.

In yet another preferred embodiment, the first time of flight is shorter than the second time of flight, i.e. the at least one reference element arranged such that the first acoustic waves impinge on the detection unit prior to the second acoustic waves. This is particularly advantageous because the first detection signal, which is used preferably as a reference signal for calibrating the device, is available to the processing unit prior to the second detection signal. By this means, the first detection signal can be prepared in advance for the correction of the second detection signal, e.g. by weighting or a spectral analysis, thus speeding up the correction process.

In yet another preferred embodiment, the detection unit has a detection bandwidth in which the detection unit is sensitive to acoustic waves of different frequencies, the detection bandwidth having a center frequency and a corresponding center wavelength λ_{c}, and the at least one reference element having a size (d), in particular a diameter, corresponding to 0.5 to 1.5 times the center wavelength λ_{c}: d=a λ_{c}, wherein a=0.5 to 1.5. In particular, the radius of the reference element, e.g. a sphere fabricated at least partially of an electromagnetic radiation absorbing material, can be adjusted to the center frequency leading to an emission of e.g. a spherical wave with a wavelength corresponding to a peak sensitivity of the detection unit. By this means, the SNR can be reliably maximized.

In yet another preferred embodiment, the detection unit comprises a sensitivity field and is configured to detect acoustic waves within the sensitivity field. Preferably, the at least one reference element is arranged at least partially within the sensitivity field of the detection unit. Alternatively, the at least one reference element is arranged at least close to the sensitivity field, in particular adjacent to the sensitivity field. For example, the at least one reference element may be located close to, preferably at least partially within, an area on the surface of the object through which the second acoustic waves pass when propagating, in particular along the second propagation path, towards the detection unit such that the first acoustic waves and the second acoustic waves impinge on the detection unit substantially under the same angle. Alternatively or additionally, the sensitivity field exhibits a focus point, wherein the at least one reference element is at least partially arranged in the focus point or at least adjacent to the focus point. By this means, it is achieved that at least a significant part of the first acoustic waves generated by the reference element contributes to the first detection signal, i.e. a reference signal, thus allowing a reliable calibration of the device.

Preferably, the at least one reference element has its radius adapted to the peak detection frequency of the detection unit at the reference element position. Common detection units utilized in optoacoustic imaging are physically focused at least in one dimension, leading to a spatially frequency dependent detection sensitivity, where peak detection sensitivity at the center frequency typically resides in the sensitivity field and decreases with the distance to the sensitivity field. Lower frequency signals encounter a bigger area of sensitivity—sometimes also termed out-of-plane signal. This effect can be utilized by providing at least one reference element comprising a size, in particular radius, as a function of the distance of the at least one reference element to the sensitivity field, in particular to a lateral distance of the at least one reference element to an axis of symmetry of the detection unit, thus increasing the acoustic energy of the emitted acoustic waves on the one hand, but also increasing the resulting amplitude of the detection signal due to the matching of the signals frequency to the detector sensitivity.

In yet another preferred embodiment, the at least one reference element comprises at least one first reference element and at least one second reference element, the at least one first reference element being arranged such that a first part of the electromagnetic radiation emitted by the irradiation unit directly impinges on the at least one first reference element, and the at least one second reference element being arranged such that a second part of the electromagnetic radiation, after having been at least partially reflected and/or scattered by the object, indirectly impinges on the at least one second reference element. The detection unit is preferably configured to detect first acoustic waves emitted by the at least one first reference element and the at least one second reference element and to generate a first detection signal of the first reference element and a first detection signal of the second reference element, respectively. The processing unit is preferably configured to correct the first detection signal of the first reference element using the first detection signal of the second reference element to obtain a corrected first detection signal, to correct, in particular to normalize, the second detection signal using the corrected first detection signal to obtain the corrected second detection signal, and to generate the image information regarding the object based on the corrected, in particular normalized, second detection signal. This can help to remove the influence of reflected electromagnetic radiation, which is dependent on the surface and properties of the object and would otherwise contribute to the first detection signal, i.e. the reference signal. Thus, the reliability regarding calibration of the device can be substantially increased using the knowledge about the influence of the amount of radiation reflected by the object.

Preferably, the at least one second reference element is arranged outside of an illumination cone inside which the electromagnetic radiation propagates from the irradiation unit towards the object to reliably prevent direct illumination of the at least one second reference element.

Advantageously, the first acoustic waves emitted by the at least one second reference element contain information regarding the influence of scattered light on the generation of first acoustic waves. Accordingly, the corresponding first detection signal(s) of the at least one second reference element may preferably be used to correct the first detection signal of the first reference element, which can in turn be used to calibrate the device, in particular the detection unit, e.g. by normalizing the second detection signal of the object.

Alternatively or additionally, the corresponding first detection signal(s) of the at least one second reference element may preferably be used to adapt the intensity and/or energy of the emitted electromagnetic radiation and/or to adapt the processing of the second detection signal. In this way, an adaptation of the irradiation and/or signal processing to the absorption characteristics of the object, e.g. the skin of a patient such as a brighter or darker skin type, is possible.

Alternatively or additionally, the corresponding first detection signal(s) of the at least one second reference element may preferably be used to detect if the device, in particular a transparent wall section such as a membrane of a coupling medium compartment of the device, is not in appropriate contact with the object. In this way, adverse effects of inappropriate contact with the object on the image quality and/or laser safety can be eliminated or at least reduced.

Further, the at least one second reference element is preferably arranged such that the first time of flight, which is required by the first acoustic waves emitted by the at least one second reference element to impinge on the detection unit is different to both the first time of flight, which is required by the first acoustic waves emitted by the at least one first reference element to impinge on the detection unit and the second acoustic waves emitted by the object to impinge on the detection unit. In particular, the first time of flight associated with the second reference element is longer than the first time of flight associated with the first reference element and shorter than the second time of flight. Alternatively, the first time of flight associated with the second reference element is shorter than both the first time of flight associated with the first reference element and the second time of flight. By this means first detection signals of the first reference element, first detection signals of the second reference element and second detection signals can be easily and reliably discerned from one another.

In yet another preferred embodiment, the detection unit comprises a detection surface which is sensitive to acoustic waves impinging on the detection surface, wherein the at least one second reference element is provided on the detection surface and/or formed by the detection surface. Especially the commonly used piezoelectric detectors typically rely on a polymer based matching layer which can entail some optical absorption, giving rise to optoacoustic signals on the detection surface—in the literature this is often termed “first arriving signal”—that is dependent on the incident excitation energy. The acoustic wave created will be detectable by the detection unit. Thus, the detection surface may be used as an optoacoustic emitter to quantify the amount of electromagnetic radiation backscattered at the surface of the object. By this means a particularly reliable corrected first detection signal may be obtained.

Because of the proximity of the at least one second reference element to the detection unit, this is particularly advantageous in scenarios where there is substantially no coupling medium, i.e. where the detection unit is contacting the surface of the object.

In yet another preferred embodiment, the device further comprises a compartment containing a coupling medium, the compartment and/or coupling medium being configured to acoustically couple the detection unit to the object, the compartment comprising at least one lateral wall section and at least one distal wall section, at least a part of the distal wall section being transparent to the electromagnetic radiation emitted by the irradiation unit and to acoustic waves emitted by the object, wherein the at least one reference element is provided in the coupling medium and/or in or at the at least one lateral wall section and/or in or at the at least one distal wall section. Thus, the propagation path, in particular the primary propagation path, preferably runs through the compartment, in particular the coupling medium, in particular from the distal wall section to the detection unit opposite of the distal wall section. Accordingly, the first detection signal preferably contains information relating to the coupling medium, in particular to the sound velocity in the coupling medium and/or the attenuation of the emitted electromagnetic radiation and/or acoustic waves in the coupling medium. By this means, the first detection signal contains particularly comprehensive information regarding the properties of components of the device and/or environmental conditions.

Additionally, the gain or detection sensitivity of the detection system may be temporally adjusted to optimize the detection of the reference signal. Depending on the absorption properties and size of the reference emitter, the factor applied to the amplification lies between 0.1 and 3.

In some embodiments, the at least one reference element is a spherical or semispherical object of the same material as the coupling medium comprising (optical) scattering and absorption means, e.g. TiO2-particles, india ink, nigrosin or another colorant. In particular, the at least one reference element may comprise an intralipid. Said additives can be used to easily and reliably adjust its optical properties.

Alternatively or additionally, the reference element comprises a thin layer of optically absorbing material such as a colored foil (e.g. PTFE, aluminum) or a layer of paint supported by the at least one lateral wall section and/or the distal wall section of the compartment. In this case the reference element preferably emits a wave planar with respect to the supporting wall, in particular at a frequency relating to the thickness of the layer. An advantage of this configuration is the ease of manufacturing of such an absorber, as well as the inherent advantage that the emitted wave will correspond to the interface of the paint layer and the compartment wall. Due to its transient nature and the suboptimal acoustic wave guide property of the compartment walls, detection of the acoustic wave emitted on the side of the thin layer facing the supporting wall is substantially suppressed.

In yet another preferred embodiment, the at least one reference element has a first acoustic impedance, and the coupling medium, the at least one lateral wall section and/or the at least one distal wall section having a second acoustic impedance, the first impedance and second impedance being substantially identical or at least similar. By this means, attenuation of the first and second or second acoustic waves biasing, in particular distorting, the first and second detection signal can be prevented or at least be reduced.

In yet another preferred embodiment, the at least one reference element is configured, in particular arranged and/or shaped, such that the total size of the surface of the reference element facing towards the detection unit is, in particular considerably, larger than the total size of the surface of the reference element facing towards the object. For example, the surface facing towards the detection unit comprises a substantially spherical shape whereas the surface facing towards the object comprises a substantially planar shape. Alternatively or additionally, at least a part of the surface facing towards the detection unit may comprise spikes and/or indentations. In particular, said part of the surface may be ragged, e.g. saw-toothed. By this means, most of the first acoustic waves emitted by the at least one reference element are emitted towards the detection unit such that the amount of scattered, in particular reflected, first acoustic waves impinging on the detection unit is considerably minimized, further enhancing the SNR.

For example, a spherical first section of the surface of the at least one reference element is oriented towards the detection unit, and a non-spherical second section of the surface points in a direction that does not allow multi-path propagation towards the detection unit, e.g. by means of reflection. Such multi-path propagation would arrive at the detection unit at a later time and would interfere with optoacoustic signals emitted by the object, thus causing image artefacts. For example, the non-spherical second section of the surface may be oriented towards a section of compartment comprising acoustic traps to minimize the impact on image quality.

In yet another preferred embodiment, the device further comprises an acoustic trap arrangement configured to absorb acoustic waves impinging on the acoustic trap arrangement, wherein the acoustic trap arrangement is arranged such that at least a part of the first acoustic waves and/or second acoustic waves, which are not emitted towards the detection unit, impinge on the acoustic trap arrangement. For example, the acoustic trap arrangement is arranged at least partially on the at least one lateral side wall section of the compartment. Alternatively or additionally, the acoustic trap arrangement is arranged between the at least one reference element and the object such that first acoustic waves emitted from the at least one reference element towards the object are absorbed instead of being reflected at the surface of the object and subsequently detected, in particular simultaneously with the second acoustic waves. By this means the signal to noise ratio of the first and second detection signals can be increased even further.

In yet another preferred embodiment, the device further comprises a sensor unit configured to detect at least one operational parameter of the device, in particular at least one property, e.g. the intensity and/or energy, of the electromagnetic radiation emitted by the irradiation unit, and to generate an according sensor signal, wherein the processing unit is further configured to derive information regarding a condition of the device, in particular regarding a condition of a coupling medium contained in a compartment of the device, based on the first detection signal and the sensor signal. In this way, in addition to calibrating the device, a diagnosis of the device, e.g. regarding a possible degradation of the device or its components during operation, is enabled.

For example, if the sensor signal corresponding to the detected intensity of the electromagnetic radiation does not exhibit a relevant decrease with respect to the sensor signal or intensity, respectively, at the begin of the operation, and the first detection signal exhibits a decrease with respect to the begin of the operation, it can be concluded that the decrease of the first detection signal is very likely resulting from a degradation of the coupling medium and/or another component of the device located between the object and/or reference element(s) and the detection unit rather than from a degradation of the irradiation unit. On the other hand, if both the sensor signal and the first detection signal exhibit a decrease, it is likely that a degradation of the irradiation unit has occurred and is contributing to the decrease of the first detection signal.

Preferably, the processing unit is configured to generate and/or output information and/or control the device based on the derived information regarding the condition of the device.

For example, in case that a degradation of certain component(s) of the device has been diagnosed, the processing unit may stop further operation of the device and/or output information, e.g. via a display, requiring a service intervention by a user, e.g. a replacement of the coupling medium and/or maintenance of the irradiation unit.

Alternatively or additionally, the processing unit may comprise a closed-loop control unit which is configured to control the intensity and/or energy of the emitted electromagnetic radiation based on the first detection signal and/or the derived information regarding the condition of the device. In this way, an irradiation of the object with essentially the same electromagnetic radiation energy is achieved.

Alternatively or additionally, the processing unit may comprise a closed-loop control unit which is configured to control the sensitivity of the detection unit to acoustic waves, in particular to second acoustic waves, based on the first detection signal and/or the derived information regarding the condition of the device. In this way, variations of the magnitude of the detection signal, in particular the second detection signal, which are due to variations of, e.g., the energy and/or intensity of the electromagnetic energy can be eliminated or at least reduced.

Alternatively or additionally, the processing unit may comprise a protective circuit which is configured to reduce the intensity and/or energy of the emitted electromagnetic radiation and/or to stop the generation and emission of the electromagnetic radiation based on the first detection signal and/or the derived information regarding the condition of the device. For example, in case that the first detection signal obtained from the at least one reference element reveals that the energy and/or intensity of the electromagnetic radiation exceeds a predetermined value, which preferably corresponds to a laser protection class, the emission of radiation and/or irradiation of the object is stopped and/or the intensity and/or energy of the radiation is reduced. In this way, an irradiation of the object with too high electromagnetic energy can be reliably avoided.

Preferably, the aforementioned approaches of controlling the device are performed in real time.

Further advantages, features and examples of the present invention will be apparent from the following description of following figures:

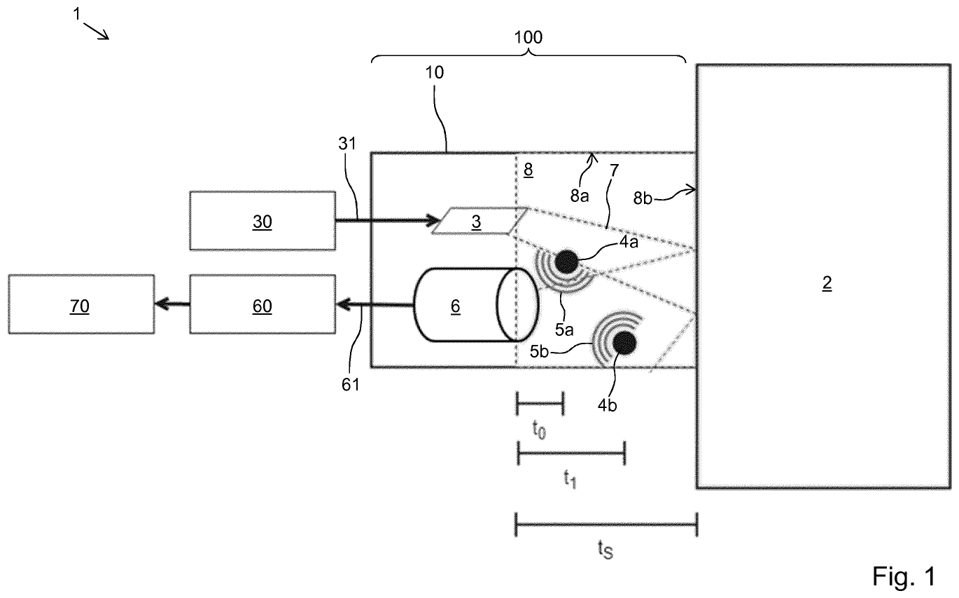

FIG. 1 shows an example of a device for optoacoustic imaging of an object comprising a first reference element and a second reference element; and

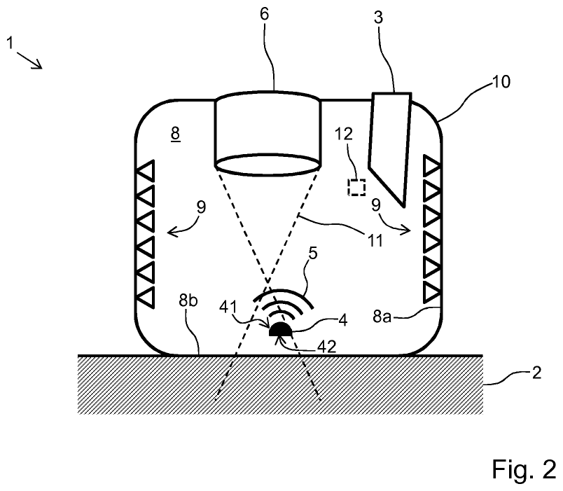

FIG. 2 shows a compartment of an exemplary device for optoacoustic imaging of an object comprising an acoustic trap arrangement.

FIG. 1 shows an example of a device 1 for optoacoustic imaging of an object 2 in a schematic representation, the device 1 comprising an irradiation unit 3 for emitting electromagnetic radiation and irradiating the object 2 therewith, a first reference element 4a and a second reference element 4b for generating first acoustic waves 5a, 5b, respectively, upon irradiation with the electromagnetic radiation, and a detection unit 6 for detecting said first acoustic waves 5a, 5b generated by the reference elements 4a, 4b as well as second acoustic waves generated the object 2 upon irradiation of the object 2 with the electromagnetic radiation.

Preferably, the irradiation unit 3 is configured to emit pulsed electromagnetic radiation, preferably a plurality of pulses of electromagnetic radiation, or continuous electromagnetic radiation exhibiting a varying amplitude and/or frequency. For example, the irradiation unit 3 may comprise at least one laser which emits, preferably non-ionizing, laser light or a radiofrequency generator which emits radiofrequency radiation.

The device 1 further comprises a housing 10 which forms a, in particular handheld, probe 100 which is designed for being brought into contact with the object 2, for example human tissue, in order to obtain optoacoustic images, in particular 3D tomographic images, from a region of interest in the object 2. The probe 100 or the housing 10, respectively, is coupled to a radiation source 30, e.g. a laser source, and detection electronics 60, e.g. an amplifier, which in turn are coupled to a processing unit 70, e.g. a computer.

In particular, the irradiation unit 3 is coupled via a light guide 31, e.g. an optical fiber, to the radiation source 30 such that electromagnetic radiation generated by the radiation source 30 is emitted by the irradiation unit 3. For example, the irradiation unit 3 comprises an optics assembly, e.g. one or more lenses, gratings and/or the like, which generate a, preferably divergent, radiation cone 7 of electromagnetic radiation propagating towards the object 2. Alternatively, the irradiation unit 3 may be simply formed by a distal end of the light guide 31.

The detection unit 6, e.g. a single ultrasonic transducer or an ultrasonic transducer array, for instance arranged on an arc or on a straight line, is coupled to the detection electronics 60 via electric wiring 61. In response to detecting the first acoustic waves 5a, 5b emitted by the reference elements 4a, 4b and the second acoustic waves emitted by the object 2, the detection element 6 generates corresponding first and second detection signals, respectively. The detection signals are subsequently processed, e.g. amplified, by the detection electronics 60 and provided to the processing unit 70, which then corrects the second detection signal using the first detection signals and generates image information regarding the object 2 based on the corrected second detection signal.

Additionally, the detection electronics 60 may be configured to control the detection unit 6 to excite the object 2 using ultrasonic pulses at controlled points in time to enable interleaved pulse/echo ultrasound measurements, wherein the information obtained by these measurements may be additionally used by the processing unit 70 for correction of the second detection signals.

It is further possible to provide a radiation source 30 which is configured to emit electromagnetic radiation at multiple wavelengths as well as reference elements 4a, 4b which exhibit a specific optoacoustic spectrum. This allows for calibrated measurements also in multispectral imaging.

The light guide 31 and the electric wiring 61 preferably form or are at least part of an interface for connecting the probe 100 to external components of the device 1, in particular the radiation source 30 and/or the processing unit 70.

The device 1 further comprises a compartment 8 containing a coupling medium for coupling the detection unit 6 acoustically to the object 2. The coupling medium may comprise water, in particular heavy water, or a coupling gel, thereby being transparent for the electromagnetic radiation and having substantially the same index of refraction as the object 2. The coupling medium is enclosed by at least one, preferably rigid, lateral wall section 8a and at least one distal wall section 8b opposite of the irradiation unit 3 and the detection unit 6. The at least one distal wall section 8b, e.g. formed by a membrane, is preferably, at least partially, flexible to adapt its shape to the surface of the object 2. Further, the at least one distal wall section 8b is preferably at least partially transparent to the emitted electromagnetic radiation and the second acoustic waves generated in the object 2.

The first and second reference elements 4a, 4b are arranged inside the compartment 8, in particular inside the coupling medium, wherein the first reference element 4a is arranged inside the compartment 8 such that at least part of the electromagnetic radiation emitted by the irradiation unit 3 directly impinges on the first reference element 4a. In particular, the first reference element 4a is arranged at least partially inside the radiation cone 7 formed by the electromagnetic radiation propagating towards the object 2.

Because the first reference element 4a is positioned closer to the detection unit 6 with respect to the object 2, a first time of flight t₀ of the first acoustic waves 5a emitted by the first reference element 4a required to impinge on the detection unit 6 is shorter than a second time of flight t_{S} of the second acoustic waves emitted by the object 2. This is indicated in FIG. 1 by the length of lines below the compartment 8. By providing distinct first and second times of flight t₀, t_{S}, e.g. by arranging the first reference element 4a accordingly, the corresponding first and second detection signals generated by the detection unit 6 can be distinguished reliably, and accordingly the first detection signals can be easily used to correct the second detection signals by the processing unit 70.

In contrast to the first reference element 4a, the second reference element 4b is arranged inside the compartment 8 such that the emitted electromagnetic radiation cannot impinge directly onto the second reference element 4b. In particular, the second reference element 4b is positioned outside of the radiation cone 7. However, the second reference element 4b may be irradiated indirectly, i.e. by at least a part of the electromagnetic radiation which is scattered and/or reflected inside the compartment 8 and/or by the object 2. For example, a part of the electromagnetic radiation propagating towards the object 2 may be reflected at the surface of the object 2 and subsequently impinge on the second reference element 4b.

Because the first reference element 4a is positioned closer to the detection unit 6 than the second reference element 4b, a first time of flight t₁ of the first acoustic waves 5b emitted by the second reference element 4b required to impinge on the detection unit 6 is longer than the first time of flight t₀ of the first acoustic waves 5a emitted by the first reference element 4a. However, the second reference element 4b is positioned still closer to the detection unit 6 than the object 2 such that the first time of flight t₁ of the first acoustic waves 5b emitted by the second reference element 4b is shorter than the second time of flight t_{S} of the acoustic waves emitted by the object 2. Thereby, first detection signals associated with the second reference element 4b can be easily distinguished from the first detection signals associated with the first reference element 4a as well as the second detection signals associated with the object 2. Thus, the processing unit 70 may be configured to generate corrected first detection signals associated with the first reference element 4a using first detection signals associated with the second reference element 4b.

For example, the first acoustic waves 5a emitted by the first reference element 4a contain information regarding properties of the device 1, in particular regarding intensity fluctuations of the emitted electromagnetic radiation and/or attenuation of acoustic waves in the coupling medium due to temperature fluctuations. However, this information may be distorted by, in particular through superposition with, acoustic waves emitted by the first reference element 4a in response to impinging scattered electromagnetic radiation, e.g. biasing the amplitude and/or phase of the first acoustic waves 5a.

Because the first acoustic waves 5b emitted by the second reference element 4b contain information regarding the influence of scattered light on the generation of first acoustic waves 5a, 5b, the corresponding first detection signal of the second reference element 4b may be used to correct the first detection signal of the first reference element 4a, which can in turn be used to calibrate the device 1, in particular the detection unit 6, e.g. by normalizing the second detection signal of the object 2.

In an alternative to the example shown in FIG. 1, the second reference element 4 may be arranged closer to the detection unit 6 than the first reference element 4a such that the first time of flight t₁ of the first acoustic waves 5b emitted by the second reference element 4b is shorter than both the first time of flight t₀ of the first acoustic waves 5a emitted by the first reference element 4a and the second time of flight t_{S} of second acoustic waves emitted by the object 2. For example, the second reference element 4b may be arranged on a detection surface of the detection unit 6 sensitive to acoustic waves, in particular formed by said detection surface.

Preferably, the reference elements 4a, 4b have fixed positions and/or orientations with respect to the detection unit 6 and/or the irradiation unit 3 and/or the object 2. Alternatively or additionally, the reference elements 4a, 4b have fixed positions between the detection unit 6 and/or the irradiation unit 3, on the one hand, and the object 2 to be imaged, on the other hand. Preferably, the first reference element 4a is arranged in a, preferably fixed, position, such that a first part (see lower part of cone 7) of the electromagnetic radiation emitted by the irradiation unit 3 impinges on the first reference element 4a, while a second part (see larger upper part of cone 7) of the of the electromagnetic radiation emitted by the irradiation unit 3, preferably simultaneously or substantially simultaneously, impinges on the imaged object 2. Preferably, the reference elements 4a, 4b are integrated in the housing 10 or are an integral component of the probe 100. Advantageously, both first acoustic waves 5a emitted by the first reference element 4a and second acoustic waves emitted by the imaged object 2 are generated, preferably simultaneously or substantially simultaneously, during optoacoustic imaging of the object 2. As a result, first acoustic waves and according first detection signals can be detected and obtained, respectively, and used to correct the second detection signals during the optoacoustic imaging process. Advantageously, correcting the second detection signals obtained from the second acoustic waves emitted by the imaged object is not limited to or subject to a separate calibration process performed prior to or after the optoacoustic imaging process or during an interruption of the optoacoustic imaging process. Rather, it is possible to generate and detect first acoustic waves emitted by the reference element(s) whenever it is desired or necessary during the imaging process without interrupting same. For example, in case that the irradiation unit 3 emits a series and/or a plurality of pulses of electromagnetic radiation it is possible to obtain first detection signals in response to each or at least a part of the emitted pulses and to use the obtained first detection signals for correcting respective second detection signals obtained for each or at least a part of the emitted pulses.

FIG. 2 shows a schematic representation of a compartment 8 of an exemplary device 1 for optoacoustic imaging of an object 2 comprising an acoustic trap arrangement 9 for absorbing acoustic waves, wherein the compartment 8 is formed by a housing 10 which can, for example, form a handheld probe. The device 1 further comprises an irradiation unit 3 for generating electromagnetic radiation and irradiating the object 2 with the generated electromagnetic radiation, a reference element 4 for generation of first acoustic waves 5 upon irradiation with the electromagnetic radiation, and a detection unit 6 for detection of the first acoustic waves 5 and second acoustic waves generated in the object 2 upon irradiation with the electromagnetic radiation.

The detection unit 6, in particular a detection element like an ultrasonic transducer or an array of transducers, preferably comprises a sensitivity field 11, wherein the detection unit 6 primarily detects acoustic waves from within the sensitivity field 11, i.e. waves having their origin within the sensitivity field 11. Hence, the reference element 4 is preferably at least partially arranged inside the sensitivity field 11 such that the first acoustic waves 5 propagate on a primary propagation path directly from the reference element 4 to the detection unit 6. First detection signals generated by the detection unit 6 in response to the detected first acoustic waves 5 therefore characterize the primary propagation path, i.e. contain information regarding e.g. attenuation of acoustic waves along the primary propagation path.

The reference element 4 is preferably arranged close to the object 2, in particular at or on a distal wall section 8b of the compartment 8 contacting the object 2. In an alternative to the example shown in FIG. 2, the reference element 4 may be arranged outside of the compartment 8, in particular directly on the surface of the object 2. Thereby, the primary propagation path substantially corresponds to the secondary propagation path along which second acoustic waves emitted by the object 2 propagate, and the information contained in the first detection signal characterizing the primary propagation therefore may also be applied to a second detection signal generated by the detection unit 6 upon detection of the second acoustic waves. Thus, a calibration of the device 1 can be achieved, and an accordingly high quality image can be reconstructed from the (corrected) second detection signal.

To minimize the attenuation e.g. of the second acoustic waves, the reference element 4 is fabricated from the same material as a coupling medium contained inside the compartment 8 for acoustically coupling the detection unit 6 to the object 2. In this way, the reference element 4 is acoustically well matched to the coupling medium. By providing e.g. particles in the reference element 4, absorbance of the electromagnetic radiation can be achieved at the same time. For example, by providing TiO2, india ink, nigrosin or another colorant in the reference element 4, an absorption of the order of blood vessels at the wavelengths emitted by the irradiation unit 3 can be achieved, which optimizes signal to noise ratio (SNR) while not saturating the detection unit 6.

Besides first acoustic waves 5 of interest that are detected on the direct (primary) propagation path, any reference element 4 introduced into the compartment 8 may as well emit interfering signals resulting from multi-path propagation. Advantageously, those multi-path propagated signals are minimized, as they typically have detrimental impact on the image quality, since they will very likely interfere with second acoustic waves emitted by the object 2. A factor enabling multi-path propagation in the compartment 8 of a device 1 is the material and shape of the housing 10, and very specifically the interface between housing 10 and the coupling medium contained by the compartment 8. Its properties are relevant both with regard to the reflection of acoustic waves, as well as the generation of acoustic waves upon irradiation with the electromagnetic radiation. Most materials of sufficient mechanical robustness and at the same time featuring a density compatible with a handheld probe feature some optical absorbance, and will as a result emit acoustic waves upon irradiation. In the interest of image quality, such parasitic waves hitting the detection unit 6 are minimized.

To this end, preferably acoustic trap arrangements 9 are disposed inside the compartment 8, e.g. on lateral wall sections 8a of the compartment 8. The concept of an acoustic trap arrangement 9 encompasses a surface that is shaped in such a way that both generated acoustic waves as well as impinging acoustic waves are directed in a direction where they cannot be picked up by the detection unit, or can only picked up with a delay that makes them arrive after the last portion of second acoustic waves generated by the object 2 and thereby distinguishable. With regards to the optoacoustic emission it is beneficial to minimize the surface of the housing 10 that faces towards the detection unit 6, which can for instance be achieved by a microstructure featuring straight or curved surfaces, where none is directly facing the detection unit 6. To maximize the effectiveness, the shapes are to be specifically adapted to the spatial arrangement of reference elements 4 and the detection, unit 6 as well as the overall shape of the compartment 8. Acoustic wave simulations with tools such as k-wave Toolbox or Field II are preferred to derive these optimal geometries. It is furthermore preferred to maximize the acoustic absorbance of the housing 10, e.g. by choosing its material accordingly, in order to minimize the reflection of acoustic waves. The housing 10 can for instance encompass a lower acoustic impedance than the coupling medium, for example by using materials utilized in so-called anechoic (echoless) chambers.

Alternatively or additionally, the reference element 4 comprises a surface, a first section 41 of which faces the detection unit 6. Preferably, the first section 41 exhibits a larger size than a second section 42 of the surface facing the object 2.

In the given example, the first section 41 facing the detection unit 6 forms substantially a hemisphere, whereas the second section 42 facing the object 2 forms substantially a plane. By increasing the surface area of the first section 41 with respect to the second section 42, a larger part of the emitted first acoustic waves 5 will propagate along the primary propagation path towards the detection unit 6, and a smaller part the first acoustic waves 5 will be reflected at the surface of the object 2, in particular at the distal wall section 8b of the compartment 8, or at the lateral wall sections 8a of the compartment 8, further reducing the impact of parasitic waves.

Alternatively or additionally, reflection of at least a part of the first acoustic waves 5 emitted towards the object 2 can be suppressed or at least reduced by providing an acoustic reflector, in particular an acoustic trap arrangement 9, between the reference element 4 and the object 2 (not shown).

Additionally or alternatively to calibrating the device 1, a diagnosis of the device 1, e.g. regarding a possible degradation of the device 1 or its components during operation, may be provided. Preferably, a sensor unit 12 may be provided which is configured to detect at least one operational parameter of the device 1, in particular at least one property, e.g. the intensity and/or energy, of the electromagnetic radiation emitted by the irradiation unit 3, and to generate an according sensor signal. The processing unit 70 (see FIG. 1) is preferably further configured to derive information regarding a condition of the device 1, in particular regarding a property and/or condition of the coupling medium contained in the compartment 8, based on the first detection signal and the sensor signal.

For example, if the sensor signal corresponding to the detected intensity of the electromagnetic radiation does not exhibit a relevant decrease with respect to the sensor signal or intensity, respectively, at the begin of the operation, and the first detection signal exhibits a decrease with respect to the begin of the operation, it can be concluded that the decrease of the first detection signal is very likely resulting from a degradation of the coupling medium contained in the compartment 8 and/or another component of the device 1 located between the object 2 and/or reference element(s) 4, 4a, 4b (see also FIG. 1) and the detection unit 6 rather than from a degradation of the irradiation unit 3. On the other hand, if both the sensor signal and the first detection signal exhibit a decrease, it is likely that a degradation of the irradiation unit 3 has occurred and is contributing to the decrease of the first detection signal.

Preferably, the processing unit 70 is configured to generate and/or output information and/or control the device 1 based on the derived information regarding the condition of the device 1. For example, in case that a degradation of certain component(s) of the device 1 has been diagnosed, the processing unit 70 may stop further operation of the device 1 and/or output information, e.g. via a display, requiring a service intervention by a user, e.g. a replacement of the coupling medium and/or maintenance of the irradiation unit 3.

1. A device for optoacoustic imaging of an object, the device comprising:
an irradiation unit configured to emit electromagnetic radiation and to irradiate the object with the electromagnetic radiation,
at least one reference element arranged such that a part of the electromagnetic radiation emitted by the irradiation unit impinges on the reference element, the reference element being configured to emit first acoustic waves in response to the impinging electromagnetic radiation,
a detection unit configured to detect first acoustic waves emitted by the reference element and second acoustic waves emitted by the object in response to irradiating the object with the electromagnetic radiation and to generate an according first detection signal and second detection signal, respectively,
a processing unit configured to correct, in particular to normalize, the second detection signal using the first detection signal to obtain a corrected second detection signal, and to generate image information regarding the object based on the corrected, in particular normalized, second detection signal.

2. The device according to claim 1, wherein the at least one reference element being arranged such that a first time of flight, which is required by the first acoustic waves emitted by the reference element to impinge on the detection unit, is different to a second time of flight, which is required by the second acoustic waves emitted by the object to impinge on the detection unit.

3. The device according to claim 1, wherein the first time of flight being shorter than the second time of flight.

4. The device according to claim 1, wherein the detection unit having a detection bandwidth in which the detection unit is sensitive to acoustic waves of different frequencies, the detection bandwidth having a center frequency and a corresponding center wavelength (λ_{c}), and the at least one reference element having a size, in particular a diameter, corresponding to 0.5 to 1.5 times the center wavelength (λ_{c}): d=a λ_{c}, wherein a=0.5 to 1.5.

5. The device according to claim 1, wherein the detection unit comprises a sensitivity field and is configured to detect acoustic waves within the sensitivity field, and the at least one reference element is arranged at least partially within the sensitivity field of the detection unit.

6. The device according to claim 1, wherein the at least one reference element comprising at least one first reference element and at least one second reference element,
the at least one first reference element being arranged such that a first part of the electromagnetic radiation emitted by the irradiation unit directly impinges on the at least one first reference element, and
the at least one second reference element being arranged such that a second part of the electromagnetic radiation, after having been at least partially reflected and/or scattered by the object, indirectly impinges on the at least one second reference element,
wherein
the detection unit is configured to detect first acoustic waves emitted by the at least one first reference element and the at least one second reference element and to generate a first detection signal of the first reference element and a first detection signal of the second reference element, respectively, and
the processing unit is configured to correct the first detection signal of the first reference element using the first detection signal of the second reference element to obtain a corrected first detection signal, to correct, in particular to normalize, the second detection signal using the corrected first detection signal to obtain the corrected second detection signal, and to generate the image information regarding the object based on the corrected, in particular normalized, second detection signal.

7. The device according to claim 6, wherein the detection unit comprising a detection surface which is sensitive to acoustic waves impinging on the detection surface, wherein the at least one second reference element is provided on the detection surface and/or formed by the detection surface.

8. The device according to claim 1, wherein the device further comprising a compartment containing a coupling medium, the compartment and/or coupling medium being configured to acoustically couple the detection unit to the object, the compartment comprising at least one lateral wall section and at least one distal wall section, at least a part of the distal wall section being transparent to the electromagnetic radiation emitted by the irradiation unit and to acoustic waves emitted by the object, wherein the at least one reference element is provided in the coupling medium and/or in or at the at least one lateral wall section and/or in or at the at least one distal wall section.

9. The device according to claim 8, wherein the at least one reference element having a first acoustic impedance, and the coupling medium, the at least one lateral wall section and/or the at least one distal wall section having a second acoustic impedance, the first impedance and second impedance being substantially identical or at least similar.

10. The device according to claim 1, wherein the at least one reference element being configured, in particular arranged and/or shaped, such that the total size of the surface of the reference element facing towards the detection unit is, in particular considerably, larger than the total size of the surface of the reference element facing towards the object.

11. The device according to claim 1, further comprising an acoustic trap arrangement configured to absorb acoustic waves impinging on the acoustic trap arrangement, wherein the acoustic trap arrangement is arranged such that at least a part of the first acoustic waves and/or second acoustic waves, which are not emitted towards the detection unit, impinge on the acoustic trap arrangement.

12. The device according to claim 1, further comprising a sensor unit configured to detect at least one operational parameter of the device, in particular at least one property, e.g. the intensity, of the electromagnetic radiation emitted by the irradiation unit, and to generate an according sensor signal, wherein the processing unit is further configured to derive information regarding a condition of the device, in particular regarding a condition of a coupling medium contained in a compartment of the device, based on the first detection signal and the sensor signal.

13. The device according to claim 1, wherein the at least one reference element is arranged in a, preferably fixed, position and/or orientation with respect to the detection unit and/or the irradiation unit and/or the object, such that a first part of the electromagnetic radiation emitted by the irradiation unit impinges on the at least one reference element, while a second part of the electromagnetic radiation emitted by the irradiation unit, preferably simultaneously or substantially simultaneously, impinges on the object.

14. A method for controlling a device for optoacoustic imaging of an object, comprising the steps of:
controlling an irradiation unit to emit electromagnetic radiation and to irradiate the object with the electromagnetic radiation;
controlling a detection unit to detect first acoustic waves emitted by a reference element arranged such that a part of the electromagnetic radiation emitted by the irradiation unit impinges on the reference element, the first acoustic waves being emitted by the reference element in response to the impinging electromagnetic radiation, and to generate an according first detection signal;
controlling the detection unit to detect second acoustic waves emitted by the object in response to irradiating the object with the electromagnetic radiation and to generate an according second detection signal; and
controlling a processing unit to correct, in particular normalize, the second detection signal using the first detection signal to obtain a corrected second detection signal, and to generate image information regarding the object based on the corrected, in particular normalized, second detection signal.
